(19) United States
(12) Patent Application Publication
(10) Pub. No.: US 2017/0107235 A1
(43) Pub. Date: Apr. 20, 2017

(54) PYRROLOBENZODIAZEPINE COMPOUNDS FOR TREATING LYMPHOMA

(71) Applicant: ADC PRODUCTS (UK) LIMITED, Reading (GB)

(72) Inventors: Anneliese Stell, North Mymms (GB); Ana Lara, North Mymms (GB)

(21) Appl. No.: 15/301,336

(22) PCT Filed: May 29, 2015
(86) PCT No.: PCT/GB2015/051562

§ 371 Date: Sep. 30, 2016

(30) Foreign Application Priority Data
May 30, 2014 (GB) 1409653.1

(51) Int. Cl. C07D 519/00

(52) U.S. Cl. CPC C07D 519/00

(57) ABSTRACT

A method of treating a patient suffering from lymphoma comprising administering to said patient a therapeutically active amount of a compound formula I, or pharmaceutically acceptable salt or solvate thereof, wherein: the dotted lines indicate the optional presence of a double bond between C1 and C2 or C2 and C3; R² and R³ are independently selected from —H, ═O, ═CH₂, —CN, —R, OR, halo, ═CH—R, O—SO₂—R, CO₂R and COR; R⁶, R⁷ and R⁹ are independently selected from H, R, OH, OR, SH, SR, NH₂, NHR, NRR′, nitro, Me₃Sn and halo; where R and R′ are independently selected from optionally substituted C₁₋₂alkyl, C₃₋₂₀heterocyclyl and C₅₋₂₀aryl groups; R″ is a C₃₋₁₂ alkylene group, which chain may be interrupted by one or more heteroatoms, e.g. O, S, NH, and/or aromatic rings, e.g. benzene or pyridine, and each X is independently selected from O, S, or NH; R^{2′}, R^{3′}, R^{6′}, R^{7′} and R^{9′} are all independently selected from the same lists as previously defined for R², R³, R⁶, R⁷ and R⁹ respectively.

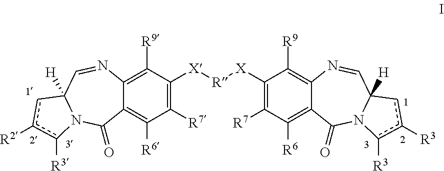

The present invention relates to pyrrolobenzodiazepine (PBD) dimer therapeutic agents useful in the treatment of lymphomas.

### BACKGROUND

### Pyrrolobenzodiazepines

Some pyrrolobenzodiazepines (PBDs) have the ability to recognise and bond to specific sequences of DNA; the preferred sequence is PuGPu. The first PBD antitumour antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and over 10 synthetic routes have been developed to a variety of analogues (Thurston, et al., Chem. Rev. 1994, 433-465 (1994); Antonow, D. and Thurston, D. E., Chem. Rev. 2011 111 (4), 2815-2864). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180 487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102)(Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)) and tomamycin (Arima, et al., J. Antibiotics, 25, 437-444 (1972)). PBDs are of the general structure:

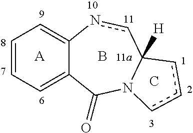

They differ in the number, type and position of substituents, in both their aromatic A rings and pyrrolo C rings, and in the degree of saturation of the C ring. In the B-ring there is either an imine (N═C), a carbinolamine(NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as antitumour agents.

A pyrrolobenzodiazepine dimer compound that is undergoing clinical trials is described by Gregson et al. (Chem. Commun. 1999, 797-798) as compound 1, and by Gregson et al. (J. Med. Chem. 2001, 44, 1161-1174) as compound 4a. This compound, also known as SG2000, is shown below:

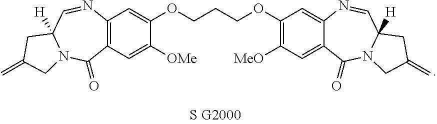

More recently the use of PBD dimers in antibody-drug conjugates has been disclosed.

### Lymphoma

Lymphomas are solid tumours of the lymphatic system. According to the U.S. National Institutes of Health, lymphomas account for about five percent of all cases of cancer in the United States, and Hodgkin's lymphoma in particular accounts for less than one percent of all cases of cancer in the United States. Because the whole lymphatic system is part of the body's immune system, patients with a weakened immune system such as from HIV infection or from certain drugs or medication also have a higher incidence of lymphoma.

Hodgkin lymphoma is one of the best-known types of lymphoma, and differs from other forms of lymphoma in its prognosis and several pathological characteristics. A division into Hodgkin and non-Hodgkin lymphomas is used in several formal classification systems. A Hodgkin lymphoma is marked by the presence of a type of cell called the Reed-Sternberg cell. There are many forms of lymphoma. Some forms of lymphoma are categorized as indolent (e.g. small lymphocytic lymphoma), compatible with a long life even without treatment, whereas other forms are aggressive (e.g. Burkitt's lymphoma), causing rapid deterioration and death. However, most of the aggressive lymphomas respond well to treatment and are curable. The prognosis therefore depends on the correct diagnosis and classification of the disease, which is established after examination of a biopsy by a pathologist (usually a hematopathologist).

Lymphoma is one of the most common malignant tumors to occur in dogs. The cause is genetic, but there are also suspected environmental factors involved. Breeds that are commonly affected include Boxer, Scottish Terrier, Basset Hound, Airedale Terrier, Chow Chow, German Shepherd, Poodle, St. Bernard, Bulldog, Beagle, Rottweiler and Golden Retriever. The Golden Retriever is especially susceptible to developing lymphoma, with a lifetime risk of 1:8 (Modiano J, et al., Cancer Res 65 (13): 5654-61. doi:10.1158/0008-5472. CAN-04-4613.)

Lymphoma is the most common malignancy diagnosed in cats. Lymphoma in young cats occurs most frequently following infection with feline leukemia virus (FeLV) or to a lesser degree feline immunodeficiency virus (FIV).

### SUMMARY OF THE INVENTION

The present invention provides dimeric PBDs that are useful in the treatment of lymphomas.

In a first aspect, the present invention relates to the treatment of a patient suffering from lymphoma, comprising administering to said patient a therapeutically effective amount of a compound of formula I:

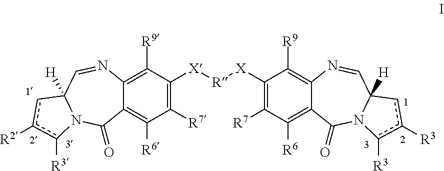

or pharmaceutically acceptable salt or solvate thereof, wherein:
the dotted lines indicate the optional presence of a double bond between C1 and C2 or C2 and C3;
R² and R³ are independently selected from —H, ═O, ═CH₂, —CN, —R, OR, halo, ═CH—R, O—SO₂—R, CO₂R and COR;
R⁶, R⁷ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R″ is a C₃₋₁₂ alkylene group, which chain may be interrupted by one or more heteroatoms, e.g. O, S, NH, and/or aromatic rings, e.g. benzene or pyridine, and each X is independently selected from O, S, or NH;
R^{2′}, R^{3′}, R^{6′}, R^{7′} and R^{9′} are all independently selected from the same lists as previously defined for R², R³, R⁶, R⁷ and R⁹ respectively.

A second aspect provides a compound of formula I as described in the first aspect for use in the treatment of lymphoma.

A third aspect provides the use of a compound of formula I as described in the first aspect in the manufacture of a medicament for the treatment of lymphoma.

### DEFINITIONS

### Substituents

The phrase “optionally substituted” as used herein, pertains to a parent group which may be unsubstituted or which may be substituted.

Unless otherwise specified, the term “substituted” as used herein, pertains to a parent group which bears one or more substitutents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

C₁₋₁₂ alkyl: The term “C₁₋₁₂ alkyl” as used herein, pertains to a monovalent moiety obtained by removing a hydrogen atom from a carbon atom of a hydrocarbon compound having from 1 to 12 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C5), n-hexyl (C₆) and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

C₂₋₁₂ Alkenyl: The term “C₂₋₁₂ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₁₂ alkynyl: The term “C₂₋₁₂ alkynyl” as used herein, pertains to an alkyl group having one or more carbon-carbon triple bonds.

Examples of unsaturated alkynyl groups include, but are not limited to, ethynyl (ethinyl, —C≡CH) and 2-propynyl (propargyl, —CH₂—C≡CH).

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);
unsaturated monocyclic hydrocarbon compounds:
   cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and
saturated polycyclic hydrocarbon compounds:
   norcarane (C₇), norpinane (C₇), norbornane (C₇).

C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and, N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. Preferably, each ring has from 5 to 7 ring atoms.

In this context, the prefixes (e.g. C₃₋₂₀, C₅₋₇, C₅₋₆, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”.

Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl which comprise fused rings, include, but are not limited to:
C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ (with 2 fused rings) derived from benzodiazepine (N₂);
C₁₃ (with 3 fused rings) derived from carbazole (N₁), dibenzofuran (O₁), dibenzothiophene (S₁), carboline (N₂), perimidine (N₂), pyridoindole (N₂); and,
C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇alkyl group.

Alkoxy: —OR, wherein R is an alkyl group, for example, a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR¹), wherein R¹ is a hemiacetal substituent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH)(OMe) and —CH(OH)(OEt).

Ketal: —CR(OR¹)(OR²), where R¹ and R² are as defined for acetals, and R is a ketal substituent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

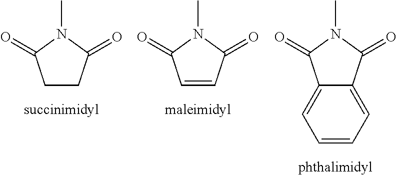

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

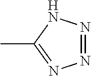

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇alkyl group. Examples of imino groups include, but are not limited to, ═NH, ═NMe, and ═NEt.

Amidine (amidino): —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Phosphino (phosphine): —PR₂, wherein R is a phosphino substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein R is a phosphinyl substituent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR)₂, where R is a phosphonate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR)₂, where R is a phosphate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR)₂, where R is a phosphite substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR¹)—NR²², where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²², where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

### Alkylene

C₃₋₁₂ alkylene: The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)n— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH—₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

### Methods of Treatment

As described above, the present invention provides the use of a compound of formula I, or a pharmaceutically acceptable salt or solvate thereof, in a method of therapy. It is preferred that the compound of formula I is administered in the form of a pharmaceutical composition.

The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of medical or veterinary doctors.

A compound of the invention may be administered alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rlL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG™, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the conjugates of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

For the avoidance of doubt, the present invention does not cover the situation where the compound of formula I is colvalently bound to an antibody or other cell binding agent.

### Lymphoma

### Lymphoma in Humans

Several classification systems have existed for human lymphoma. These systems use histological findings and other findings to divide lymphoma into different categories. The classification of lymphoma can affect treatment and prognosis. Classification systems generally classify lymphoma according to:
Whether or not it is a Hodgkin lymphoma
Whether the cell that is replicating is a T cell or B cell
The site that the cell arises from.

The current accepted definition is the WHO classification, published in 2001 and updated in 2008. This is the latest classification of lymphoma and is based upon the foundations laid within the “Revised European-American Lymphoma classification” (REAL). This system attempts to group lymphomas by cell type (i.e. the normal cell type that most resembles the tumor) and defining phenotypic, molecular or cytogenetic characteristics. There are three large groups: the B cell, T cell, and natural killer cell tumors. Other less common groups, are also recognized. Hodgkin lymphoma, although considered separately within the World Health Organization (and preceding) classifications, is now recognized as being a tumor of, albeit markedly abnormal, lymphocytes of mature B cell lineage.

The table below sets out the major types of lymphoma, as well as the drugs currently used in their treatment:

| Lymphoma | | |
|---|---|---|
| subgroup | | |
| according | | |
| to WHO | | Cytotoxic drugs/radiotherapy/surgical |
| 2008 | Lymphoma | treatment/antibiotics/antivirals |
| Mature B- | Diffuse large B cell | R-CHOP- cyclophosphamide, doxorubicin, |
| cell | lymphoma | vincristine, prednisilone |
| neoplasms | | R-ACVBP- doxorubicin, cyclophosphamide, |
| | | vindesine, bleomycin, prednisolone/other |
| | | steroids |
| | | R-CHOEP- above plus etoposide |
| | | R-CEOP- above with substitution of |
| | | doxorubicin for etoposide |
| | | R-C (liposomal doxorubicin)OP |
| | | R-DHAP- |
| | | dexamethasone/cytarabine/cisplatin |
| | | R-ICE- ifosfamide/carboplatin/etoposide |
| | Primary effusion | Sirolimus/difficult to treat with standard |
| | lymphoma | chemotherapy |
| | Burkitt's lymphoma | cyclophosphamide |
| | | vincristine |
| | | doxorubicin |
| | | methotrexate |
| | | ifosfamide |
| | | cytarabine |
| | | etoposide |
| | | steroids |
| | | rituximab |
| | Follicular lymphoma-often | R-CVP - cyclophosphamide/ |
| | indolent, when advanced | vincristine/prednisilone |
| | cytotox treatment | Chlorambucil |
| | | R-CHVP |
| | | Fludarabine |
| | | bendamustine |
| | Small lymphocytic | As above |
| | lymphoma | |
| | Marginal zone lymphoma- | Gastric MALT- |
| | extranodal | H. pylori eradication (triple antibiotics |
| | | regimen) |
| | | then if aggressive consider chemo such as |
| | | bendamustine/fludarabine/rituximab |
| | | Non gastric MALT- |
| | | radiotherapy and as for indolent lymphomas |
| | | above |
| | Marginal zone lymphoma- | Fludarabine/pentostatin |
| | nodal | R-CVP |
| | | R-CHOP |
| | Marginal zone lymphoma- | Splenectomy/splenic irradiation if not fit |
| | splenic | Chemo as for indolent lymphomas |
| | Mantle cell lymphoma | R-CHOP |
| | | Alternating methotrexate/cytarabine |
| | | Hyper-CVAD: hyperfractionated |
| | | cyclophosphamide/vincristine/doxorubicin/dexamethasone |
| | | High dose cytarabine |
| | | Bendamustine |
| | | Chlorambucil |
| | | Rituximab |
| | | Idelalisib |
| | | everolimus/temsiroli mus |
| | | bortezomib |
| Hodgkin's | Classical | BEACOPP- |
| lymphoma- | Nodular-lymphocyte | bleomycin/etoposide/doxorubicin/cyclophosphamide/ |
| subcategory | predominant | vincristine/procarbazine/prednisilone |
| of mature B | | ABVD- doxorubicin/bleomycin/vinblastine/ |
| cell | | dacarbazine |
| neoplasms | | DHAP- as previously |
| | | IGEV- ifosfamide/gemcitabine/vinorelbine |
| | | ICE- as previously |
| | | rituximab |
| Mature T/NK | Cutaneous: Mycosis | Gemcitabine |
| cell | fungoides/Transformed | Liposomal doxorubicin |
| neoplasms | MF | Denileukin-diftitox |
| | | vorinostat/romidepsin |
| | Cutaneous: Sezary | ECP- etoposide/cisplatin/prednisolone |
| | syndrome | Methotrexate |
| | | Chlorambucil |
| | | Bexarotene |
| | | Denileukin-diftitox |
| | Cutaneous: | Targeted drugs becoming more relevant e.g. |
| | Primary cutaneous | crizotinib/pralatrexate |
| | Anaplastic large cell | |
| | lymphoma | |
| | Nodal includes: | CHOP |
| | ALCL ALK+ | CHOEP |
| | ALCL ALK− | Etoposide/ifosfamide/cisplatin |
| | PTCL NOS | Gemcitabine based- e.g. GEM-P |
| | Angio-immunoblastic TCL | cisplatin/etoposide/gemcitabine/dexamethasone |
| | | Or GIFOX- gemcitabine/ |
| | | ifosphamide/oxaliplatin |
| | | Carmustine/etoposide/cytarabine/melphalan |
| | Extranodal | Radiotherapy to nose and L-asparaginase |
| | NK/T cell lymphoma nasal | |
| | type | |
| | Extranodal: subcutaneous | CHOP-like regimens |
| | panniculitis-like TCL | |

For the avoidance of doubt, B-CLL (B-cell chronic lymphocytic leukemia) is not considered a lymphoma.

In some aspects of the present invention, the human lymphoma treated is not small lymphocytic lymphoma.

In some aspects of the present invention, the human lymphoma treated is one not involving the B cell lymphocytes. Thus in these aspects, the lymphoma may be a Mature T cell or natural killer (NK) cell neoplasm.

In some aspects of the invention, treatment with or the use of a compound of formula I may be combined with any one of the know treatment regimens, or any element thereof.

Existing cytotoxic treatments can be categorised as follows:

(a) Alkylating drugs: N2 mustards, such as cyclophosphamide, melphalan, ifosfamide and chlorambucil; nitrosureas, such as carmustine and bendamustine; tetrazines, such as dacarbazine; platinums, such as cisplatin, carboplatin and oxaliplatin; Non-classical agents, such as procarbazine;
(b) Antimetabolites: antifolates, such as methotrexate; deoxyribonucleic acid analogues, such as gemcitabine′ fludarabine, cladribine and pentostatin;
(c) Topoisomerase inhibitors: topisomerase II inhibitors, such as doxorubicin and etoposide;
(d) Cytotoxic antibiotics: bleomycin;
(e) Antimicrotubule inhibitors: vincristine and vinblastine.

Newer treatments can be categorised as follows:

(a) PI3Ki inhibitors: idelalisib;
(b) mTOR inhibitors: everolimus and temsirolimus;
(c) ALK/MET inhibitor: Crizotonib;
(d) Proteasome inhibitors: bortezomib;
(e) BTK-inhibitor: ibrutinib;
(f) HDAC-inhibitors: vorinostat and romidepsin;
(g) Antibodies: Anti-CD20, such as rituximab; Anti-CD25, such as denileukin-diftitox; Anti-CD30 ADC such as brentuximab vedotin; Anti-CD52 antibody, such as Alemtuzumab;
(h) Retinoids: especially for cutaneous lymphomas, such as Bexarotene;
(i) Anti-angiogenics: Lenalinomide (thalidomide like).

### Lymphoma in Dogs

The cancer is classified into low and high grade types. Classification is also based on location. The four location types are multicentric, mediastinal, gastrointestinal, and extranodal (involving the kidney, central nervous system, skin, heart, or eye). Multicentric lymphoma, the most common type (by greater than 80 percent), is found in the lymph nodes, with or without involvement in the liver, spleen, or bone marrow. Mediastinal lymphoma occurs in the lymph nodes in the thorax and possibly the thymus. Gastrointestinal lymphoma occurs as either a solitary tumor or diffuse invasion of the stomach or intestines, with or without involvement in the surrounding lymph nodes, liver or spleen. Classification is further based on involvement of B-lymphocytes or T-lymphocytes. Approximately 70 percent are B-cell lymphoma. Cutaneous lymphoma can be classified as epitheliotropic (closely conforming to the epidermis) or non-epitheliotropic. The epitheliotropic form is typically of T-cell origin and is also called mycosis fungoides. The non-epitheliotropic form is typically of B-cell origin.

Classification of malignant lymphoma in dogs is based on anatomic location, histologic criteria, and immunophenotypic characteristics. The most common anatomic forms of lymphoma, in order of decreasing prevalence, are multicentric, gastrointestinal (GI), mediastinal, and cutaneous forms. Primary extranodal forms, which can occur in any location outside the lymphatic system, include the eyes, central nervous system (CNS), bone marrow, bladder, heart, and nasal cavity.

The World Health Organization (WHO) also publishes a histologic classification scheme, which uses the revised European American lymphoma (REAL) system as a basis for defining histologic categories of hematopoietic and lymphoid tumors in domestic animals.

| | |
|---|---|
| B-cell neoplasms | Precursor B lymphoblastic lymphoma |
| | B-cell small lymphocytic lymphoma |
| | Lymphocytic lymphoma-intermediate type |
| | Lymphoplasmacytic lymphoma |
| | Mantle cell lymphoma |
| | Follicular center cell lymphomas |
| | Marginal zone lymphoma (splenic, nodal, |
| | mucosa-associated lymphoid tissue) |
| | Plasma cell myeloma/plasmacytoma |
| | Diffuse large cell lymphoma |
| | T-cell-rich, B-cell lymphoma |
| | Large cell immunoblastic lymphoma |
| | Mediastinal (thymic) large B-cell lymphoma |
| | Burkitt's lymphoma |
| T-cell and natural | Precursor T lymphoblastic lymphoma |
| killer (NK) cell | Intestinal T-cell lymphoma |
| lymphomas | Mycosis fungoides/Sezary syndrome |
| | Cutaneous nonepitheliotropic lymphoma |
| | Anaplastic large cell lymphoma |
| | Peripheral T-cell lymphoma, unspecified |

It is important to determine the histologic grade of canine lymphomas as low (small lymphocytic or centrocytic lymphomas) or intermediate to high (diffuse large cell, centroblastic, and immunoblastic lymphomas) and the architecture as diffuse or follicular. Furthermore, determining the immunophenotype of the tumor provides useful information. The WHO classification staging system includes both Anatomic Site, as well as stage, as shown in the tables below:

Anatomic site

| | |
|---|---|
| A | Generalized |
| B | Alimentary |
| C | Thymic |
| D | Skin |
| E | Leukemia (true)* |
| F | Others (including |
| | solitary renal) |

*not part of the present invention

Stage

| | |
|---|---|
| I | Involvement limited to a single node or lymphoid tissue in a single |
| | organ.† |
| II | Involvement of many lymph nodes in a regional area (±tonsils). |
| III | Generalized lymph node involvement, |
| IV | Liver and/or spleen involvement (±stage III). |
| V | Manifestation in the blood and involvement of bone marrow and/or |
| | other organ systems (±stage I-IV). |

†Excluding bone marrow.

### Existing Treatments of Multicentric Lymphoma

The therapeutic approach to a particular patient with lymphoma is determined by the stage and substage of disease, the presence or absence of paraneoplastic disease, the overall physiologic status of the patient, financial and time commitment of the clients, and their level of comfort with respect to likelihood of treatment-related success and/or side effects. Without treatment, most dogs with lymphoma will die of their disease in 4 to 6 weeks after diagnosis, although significant variability exists. Most current treatment is with multidrug protocols, which are summarised as follows: COP; A; A+piroxicam; VMC-L; VCA-L; L-COPA; COPLA/LVP; VELCAP-SC; VLCAP-Long; L-VCAMP; L-VCAP; L-VCAP/CCNU/MOPrP; COArP; L-VCADP, wherein L, L-Asparaginase; V, vincristine; C, cyclophosphamide; M, methotrexate; Mx, mitoxantrone; O, Oncovin (vincristine); P, prednisone; A, Adriamycin (doxorubicin); D, dactinomycin; Pr, procarbazine; Ar, cytosine arabinoside.

In the context of single agent protocols, the most effective, currently available chemotherapeutic agents for canine lymphoma include doxorubicin, L-asparaginase, vincristine, cyclophosphamide, and prednisone—most of which are represented to one degree or another in most first-line multiagent chemotherapy protocols. Other drugs that have documented activity are often considered second-line agents and include lomustine, vinblastine, actinomycin-D, mitoxantrone, mustargen, chlorambucil, methotrexate, dacarbazine (DTIC), 9-aminocamptothecin, ifosfamide, cytosine arabinoside, and gemcitabine. Of these, cytosine arabinoside, ifosfamide, and gemcitabine appear to have only minimal activity. With the exception of doxorubicin, induction therapy with single-agent chemotherapy does not typically result in durable remission durations when compared with standard combination protocols.

Treatment is usually stopped on remission, with reinduction if the lymphoma recurs. If reinduction fails or the dog does not respond to the initial induction, the use of so-called “rescue” agents or “rescue” protocols may be attempted. These are single drugs or drug combinations that are typically not found in standard CHOP protocols and are withheld for use in the drug-resistant setting. The most common rescue protocols used in dogs include single-agent use or a combination of actinomycin D, mitoxantrone, doxorubicin (if doxorubicin was not part of the original induction protocol), dacarbazine (DTIC), temozolomide, lomustine (CCNU), L-asparaginase, mechlorethamine, vincristine, vinblastine, procarbazine, prednisone, and etoposide.

In some aspects of the invention, treatment with or the use of a compound of formula I may be combined with any one of the know treatment regimens, or any element thereof.

In some aspects of the present invention, the canine lymphoma treated is one not involving the B cell lymphocytes.

### Lymphoma in Cats

As mentioned above, lymphoma in young cats occurs most frequently following infection with feline leukemia virus (FeLV) or to a lesser degree feline immunodeficiency virus (FIV). These cats tend to have involvement of lymph nodes, spine, or mediastinum. Cats with FeLV are 62 times more likely to develop lymphoma, and cats with both FeLV and FIV are 77 times more likely. Younger cats tend to have T-cell lymphoma and older cats tend to have B-cell lymphoma. Older cats tend to have gastrointestinal lymphoma without FeLV infection, although tests more sensitive to low level FeLV infections and replication-defective FeLV have found that many of these cats have been previously exposed. The same forms of lymphoma that are found in dogs also occur in cats, but gastrointestinal is the most common type. Lymphoma of the kidney is the most common kidney tumor in cats, and lymphoma is also the most common heart tumor.

Gastrointestinal lymphoma is classified as low grade, intermediate grade, and high grade. Low grade types include lymphocytic and small cell lymphoma. High grade types include lymphoblastic, immunoblastic, and large cell lymphoma. Low grade lymphoma is only found in the small intestine, while large grade can commonly be found in the stomach.

The classification of lymphomas and treatments are very similar to those discussed above for dogs.

In some aspects of the invention, treatment with or the use of a compound of formula I may be combined with any one of the know treatment regimens, or any element thereof.

In some aspects of the present invention, the feline lymphoma treated is one not involving the B cell lymphocytes.

### Formulations

While it is possible for the conjugate compound to be used (e.g., administered) alone, it is often preferable to present it as a composition or formulation.

In one embodiment, the composition is a pharmaceutical composition (e.g., formulation, preparation, medicament) comprising a conjugate compound, as described herein, and a pharmaceutically acceptable carrier, diluent, or excipient.

In one embodiment, the composition is a pharmaceutical composition comprising at least one conjugate compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA), Reminqton's Pharmaceutical Sciences, 20th edition, pub. Lippincott, Williams & Wilkins, 2000; and Handbook of Pharmaceutical Excipients, 2nd edition, 1994.

Another aspect of the present invention pertains to methods of making a pharmaceutical composition comprising admixing at least one [¹¹C]-radiolabelled conjugate or conjugate-like compound, as defined herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active ingredient in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

It may be preferred that the formulation is one in which the compound of formula I is in solution, i.e. not in solid form.

### Dosage

It will be appreciated by one of skill in the art that appropriate dosages of the conjugate compound, and compositions comprising the conjugate compound, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, the active compound is administered to a human patient according to the following dosage regime: about 100 mg, 3 times daily.

In one embodiment, the active compound is administered to a human patient according to the following dosage regime: about 150 mg, 2 times daily.

In one embodiment, the active compound is administered to a human patient according to the following dosage regime: about 200 mg, 2 times daily.

However in one embodiment, the conjugate compound is administered to a human patient according to the following dosage regime: about 50 or about 75 mg, 3 or 4 times daily.

In one embodiment, the conjugate compound is administered to a human patient according to the following dosage regime: about 100 or about 125 mg, 2 times daily.

For the prevention or treatment of disease, the appropriate dosage of a drug of the invention will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 1 μg/kg to 15 mg/kg (e.g. 0.1-20 mg/kg) of molecule is an initial candidate dosage for administration to the patient, whether, for example, by one or more separate administrations, or by continuous infusion. A typical daily dosage might range from about 1 μg/kg to 100 mg/kg or more, depending on the factors mentioned above. An exemplary dosage of drug to be administered to a patient is in the range of about 0.1 to about 10 mg/kg of patient weight. For repeated administrations over several days or longer, depending on the condition, the treatment is sustained until a desired suppression of disease symptoms occurs. An exemplary dosing regimen comprises a course of administering an initial loading dose of about 4 mg/kg, followed by additional doses every week, two weeks, or three weeks of a drug. Other dosage regimens may be useful. The progress of this therapy is easily monitored by conventional techniques and assays.

### Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O—), a salt or solvate thereof, as well as conventional protected forms.

### Isomers, Salts and Solvates

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

In some embodiments, compounds of the present invention have the following stereochemistry at the C11 position:

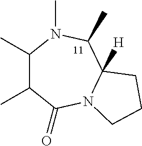

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

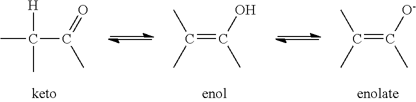

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

Solvates of particular relevance to the present invention are those where the solvent adds across the imine bond of the PBD moiety, which is illustrated below where the solvent is water or an alcohol (R^{B}OH, where R^{B} is an ether substituent as described above e.g. MeOH):

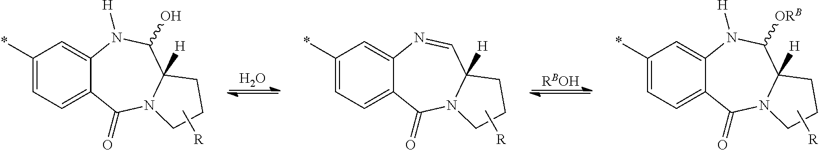

wherein * represents the dimer link to the other PBD moiety. These forms can be called the carbinolamine and carbinolamine ether forms of the PBD. The balance of these equilibria depend on the conditions in which the compounds are found, as well as the nature of the moiety itself.

In general any nucleophilic solvent is capable of forming such solvates as illustrated above for hydoxylic solvents. Other nucleophilic solvents include thiols and amines.

These solvates may be isolated in solid form, for example, by lyophilisation.

### General Synthetic Methods

The synthesis of PBD compounds is extensively discussed in the following references, which discussions are incorporated herein by reference:

a) WO 00/12508 (pages 14 to 30);
b) WO 2005/023814 (pages 3 to 10); and
c) WO 2005/085259 (pages 31 to 39).

### Further Preferences

The following preferences apply to formula I. The preferences may be combined together in any combination.

R⁹ is preferably H.

R² is preferably R, and is more preferably an optionally substituted C₅₋₂₀ aryl group or a C₁₋₇ alkyl group. Most preferred is a ═CH₂ group.

R⁶ is preferably selected from H, OH, OR, SH, NH₂, nitro and halo, and is more preferably H or halo, and most preferably is H.

R⁷ is preferably independently selected from H, OR, SH, SR, NH₂, NHR, NHRR′, and halo, and more preferably independently selected from H and OR, where R is preferably selected from optionally substituted C₁₋₇ alkyl, C₃₋₁₀ heterocyclyl and C₅₋₁₀ aryl groups. Most preferably R⁷ is OCH₃.

R″ is preferably a C₃₋₁₂ alkylene group and each X is preferably O. More preferably, R″ is a C_{3 or 5} alkylene chain and each X is O, with a R″ being C₃ propylene in the most preferable embodiments.

It is further preferred that the substituent groups on all positions of each mononmer unit that make up the dimer are the same.

In preferred aspects of the present invention, the compounds of formula I are substituted as shown in formula Ill.

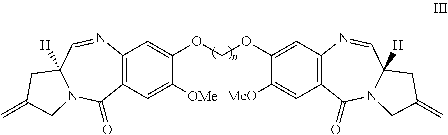

In compounds of formula Ill:

preferably n is 3 or 5;

In most preferred compounds of formula Ill, n is 3 or 5 i.e.

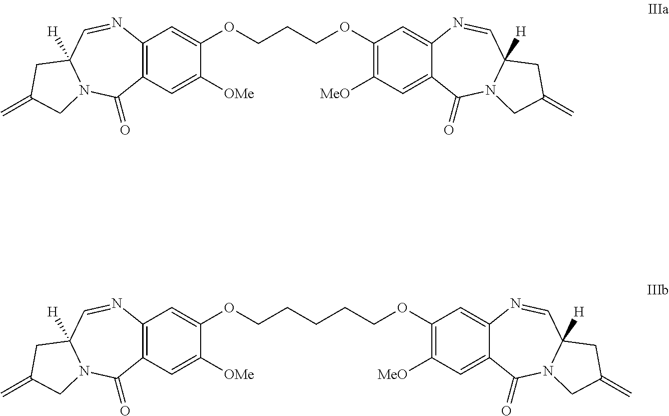

As discussed above, these compounds may be in a solvate form, for example with water or an alcohol, such as methanol, added across the imine bond:

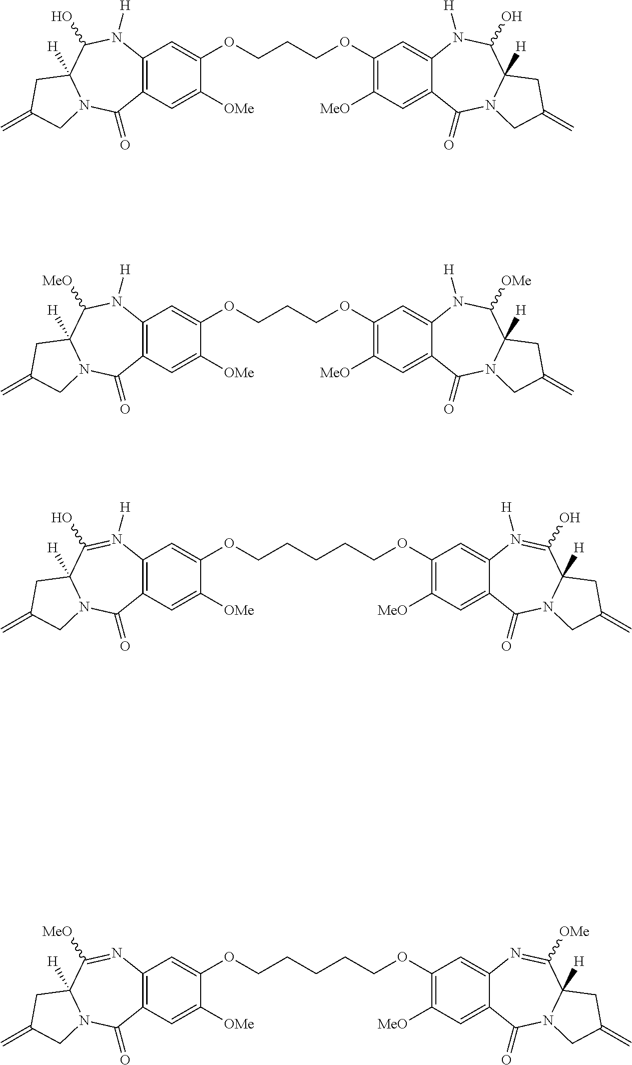

### The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus. In one preferred embodiment, the subject/patient is a human.

In another preferred embodiment, the subject is canine, for example a dog. The dog may be of one of the following breeds: Boxer, Scottish Terrier, Basset Hound, Airedale Terrier, Chow Chow, German Shepherd, Poodle, St. Bernard, Bulldog, Beagle, Rottweiler and Golden Retriever.

In another preferred embodiment, the subject is feline, for example a cat.

### EXAMPLES

A Phase Ia clinical trial in dogs with spontaneous malignancies was undertaken at the Royal Veterinary College, University of London, UK.

### Drug

### SG2000:

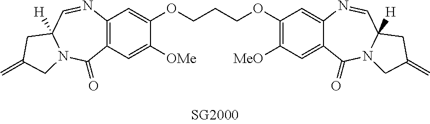

was supplied in 5 mL vials, wherein each mL contains 10 micrograms of SG2000 and 47.1 mg of dimethylacetamide. For doses below 100 microgram, the required volume of SG2000 was diluted to 10 ml with normal saline (0.9% sodium chloride) prior to administration. If the total dose exceeds 100 micrograms, the supplied solution was used without prior dilution with normal saline.

SG2000 was administered as a slow intravenous infusion into a peripheral vein. The drug was be administered slowly IV over 20 minutes via a peripheral vein catheter. If the drug has been diluted to a total volume of 10 ml, this will mean injecting 0.5 ml per minute.

The following concomitant/supportive medications permitted during the trial were:

| | |
|---|---|
| Anti-emetics | Metoclopramide, maropitant, ondansetron |
| Antacids/gastric | Famotidine, ranitidine, omeprazole, sucralfate |
| protectants | |
| Antibiotics | Metronidazole, potentiated amoxicillin etc |
| Analgesics | Methadone, butorphanol, buprenorphine, tramadol, |
| | gabapentin, amantadine, fentanyl |
| Corticosteroids | Prednisolone, dexamethasone |
| NSAIDs | Firocoxib, meloxicam, carprofen, piroxicam |
| Diuretics | Spironolactone, furosemide or other diuretics |
| Anti-histamines | Diphenhydramine |
| Anti-diarrhoeals | Loperamide |
| Appetite stimulants | Cyproheptadine |

The following definitions are used in reporting the results:

Complete response (CR) is defined as the disappearance of all target lesions.

Partial response (PR) is defined as a >30% decrease in the sum of the longest dimensions of the target lesions, taking as a reference the baseline sum longest dimensions.

Progressive disease (PD) is defined as a >20% increase in the sum of the longest dimensions of the target lesions, taking as a reference the smallest sum of the longest dimensions recorded since the treatment started, or the appearance of 1 or more new lesions.

Stable Disease (SD) is defined as neither sufficient shrinkage to qualify for PR nor sufficient increase to qualify for PD, taking as a reference the smallest sum of the longest dimensions since the treatment started.

Objective Response Rate (ORR) is defined as the number of patients with confirmed complete or partial responses, expressed as a percentage of all patients treated with SG2000

Time to Progression (TTP) is defined as the period of time from first date of treatment to the date that the patient is withdrawn because of clinical or imaging-based progressive disease, or death from any cause (including euthanasia).

Duration of Response (DR) is defined as the period of time between the first of two evaluations demonstrating an objective response until the date of progression or death. “Duration of Response” is defined for patients with an objective response only.

### Patient 1

This patient, diagnosed with multicentric B-cell high grade lymphoma, had been previously treated with 5 protocols. This patient was treated for a first cycle (3 weeks) with IV 1.75 μg/kg SG2000 solution with a single weekly dose. During this cycle the response was classified as Stable Disease (SD), but following the treatment cycle in the planned three week break the disease progressed. The second cycle of treatment followed with IV 2.00 μg/kg SG2000 with a single weekly dose. The third cycle of weekly dosing was begun after a single week break, with doses of 2.00 μg/kg SG2000, 1.75 μg/kg SG2000 and 2.00 μg/kg SG2000.

At the end of the planned 3 cycles, the patient response was classified as Progressive Disease (PD) with grade 4 anaemia. Overall, the patient's survival time from diagnosis was 408 days, with a survival time since the start of the SG2000 trial of 126 days. The progression free interval with treatment was 111 days.

### Patients 2 to 9

Patients 2 to 9 were treated as follows, with the stated amount of SG2000 (μg/kg), with a 3 weekly cycle of weekly treatments, and a single break week between cycles.

| | Cycle 1 (Dose per | | | Cycle 2 (Dose per | | | Cycle 3 (Dose per | | |
|---|---|---|---|---|---|---|---|---|---|
| Patient | therapy) | | | therapy) | | | therapy) | | |
| 2 | 1.75 | | | | | | | | |
| 3 | 1.75 | 1.75 | 1.75 | 1.75 | 1.75 | 1.75 | 2 | 1.75 | 1.75 |
| 4 | 1.75 | 2 | | | | | | | |
| 5 | 1.75 | 1.75 | 1.75 | 1.75 | 1.75 | 1.75 | 1.75 | 1.75 | 1.75 |
| 6 | 1.75 | 1.75 | 1.75 | 1.75 | 1.75 | 2 | | | |
| 7 | 1.75 | | | | | | | | |
| 8 | 1.5 | 1.5 | 1.5 | 1.5 | 1.5 | | | | |
| 9 | 1.5 | 1.5 | 1.5 | 1.5 | 1.5 | 1.5 | 1.5 | 1.5 | 1.5 |

Patients 2, 4-5 were diagnosed with multicentric B-cell high grade lymphoma, Patients 3, 8 and 9 were diagnosed with multicentric B-cell intermediate grade lymphoma and Patient 6 was diagnosed with multicentric T-cell high grade lymphoma. Patient 7 was diagnosed with multicentric and mediastinal high grade lymphoma, which was suspected to be to be T-cell lymphoma.

The following dose limiting toxicities were recorded:

| | Cycle | | |
|---|---|---|---|
| Patient | (therapy) | Dose limiting toxicities | Comment |
| 2 | 1 (1) | Grade 4 neutropenia | Advanced lymphoma |
| 3 | 1 (2) | Grade 3 proteinurea | Worsening of pre- |
| | | | existing proteinuria |
| 5 | 1 (2) | Grade 3 protein urea | Worsening of pre- |
| | | | existing proteinuria |
| | | Grade 4 liver toxicity | No evidence of |
| | | | lymphoma in liver |
| 9 | 3 (2) | Grade 4 neutropenia | |

The following outcomes were recorded:

| | | | Survival | | Protocols |
|---|---|---|---|---|---|
| | Response | Survival | time from | | failed before |
| | to | time from | SG2000 | Progression | SG2000 |
| Patient | SG2000 | diagnosis | start | free interval | treatment |
| 2 | PD | 252 d | 16 d | 10 d | 5 |
| 3 | PR | 336 d | 95 d | 54 d | 6 |
| 4 | PD | 141 d | 8 d | 7 d | 4 |
| 5 | CR | 811 d | 195 d | 158 d | 2 |
| 6 | SD | 138 d | 49 d | 43 d | 1 |
| 7 | PD | 31 d | 24 d | 10 d | 0 |
| 8 | SD | 186 d | 74 d | 56 d | 1 |
| 9 | PR | | | | 1 |

1. A method of treating a feline or a canine patient suffering from lymphoma comprising administering to said patient a therapeutically active amount of a compound of formula IIIa: 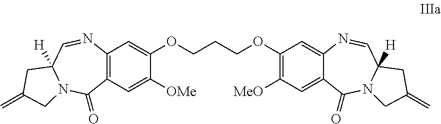
or pharmaceutically acceptable salt or solvate thereof.

2.-11. (canceled)

12. The method of claim 1, wherein the lymphoma is canine.

13. The method of claim 1, wherein the compound is in solution.

14. The method of claim 1, wherein the lymphoma treated is one not involving the B cell lymphocytes.

15. The method of claim 1, wherein the patient is a feline patient.

16. The method of claim 1, wherein the patient is a canine patient.

17. The method of claim 1, comprising administering a cancer chemotherapeutic agent.

18. The method of claim 17, wherein the cancer chemotherapeutic agent is an alkylating agent, an antimetabolite, a plant alkaloid, an antibiotic, a topoisomerase inhibitor, or a kinase inhibitor.

19. The method of claim 17, wherein the cancer chemotherapeutic agent is selected from the group consisting of: L-Asparaginase, vincristine, cyclophosphamide, methotrexate, mitoxantrone, prednisone, doxorubicin, dactinomycin, procarbazine, and cytosine arabinoside.

20. The method of claim 15, wherein the feline patient has a feline leukemia virus infection or a feline immunodeficiency virus infection.

21. The method of claim 1, wherein said administering is via intravenous administration.

22. The method of claim 1, wherein the compound is administered in a dose of from 1 μg/kg to 5 mg/kg.

23. The method of claim 1, wherein the compound is administered once weekly.
